(19) United States
(12) Patent Application Publication
(10) Pub. No.: US 2014/0340512 A1
(43) Pub. Date: Nov. 20, 2014

(54) Designs of an Automatic IV Monitoring and Controlling System

(71) Applicant: Kai Tao, Yizheng (CN)

(72) Inventor: Kai Tao, Yizheng (CN)

(21) Appl. No.: 13/897,578

(22) Filed: May 20, 2013

(51) Int. Cl. A61M 5/14; H04N 7/18

(52) U.S. Cl. CPC A61M 5/1411; H04N 7/18; A61M 2205/3306; A61M 2205/3334
USPC 348/135

(57) ABSTRACT

When analyzing video frames captured for monitoring the IV dripping process, dew droplets could exist in the image. We discussed image processing methods to remove the dew droplets from the background, including computing the difference between frames and averaging to get a proper background image. We also discussed various methods to keep the temperature of some areas of the inner surface of the drip chamber to be above the dew points in order to prevent dew droplets' formation or to remove them. In the end we showed how video of the dripping process could be shown on external display(s) for devices enclosing the drip chamber inside.

### CROSS-REFERENCE TO RELATED APPLICATIONS

U.S. Ser. No. 12/825,368 IV Monitoring by Digital Image Processing

U.S. Ser. No. 12/804,163 IV Monitoring by Video and Image Processing

U.S. Ser. No. 13/019,698 Electromechanical system for IV control

U.S. Ser. No. 13/356,632 Image Processing, Frequency Estimation, Mechanical Control and Illumination for an Automatic IV Monitoring and Controlling system

### FEDERALLY SPONSORED RESEARCH

Not Applicable

### THE NAMES OF THE PARTIES TO A JOINT RESEARCH AGREEMENT

Not Applicable

### SEQUENCE LISTING OR PROGRAM

Not Applicable

### BACKGROUND

1. Field of Intention

This invention relates to the monitoring of IV dripping process by video and image processing.

2. Prior Art

Prior art for monitoring the IV dripping processing by video and image processing means include:

CN201110955Y, application number 200710168672.3, publication date Jun. 18, 2008, Enmin Song, Huazhong University of Science & Technology, title [A medical infusion speed monitoring and controlling system]. This application outlines some general ideas, but didn't include the detailed algorithms and apparatus disclosed in our past and present applications.

Ting-Yuan Cheng, U.S. Ser. No. 12/791,885, Intravenous Drip Monitoring Method and Related Intravenous Drip Monitoring System. This application discussed some basic monitoring method based on brightness variation.

### SUMMARY

This application can be divided into three parts:

Part I: Handling of dew droplets in captured images by image processing techniques.

Part II: Techniques to prevent dew droplets' formation or removing them.

Part III: Means to display the dripping process on external display, which would otherwise not be visible to the user because the drip chamber is enclosed in the device.

### DRAWINGS

### Figures

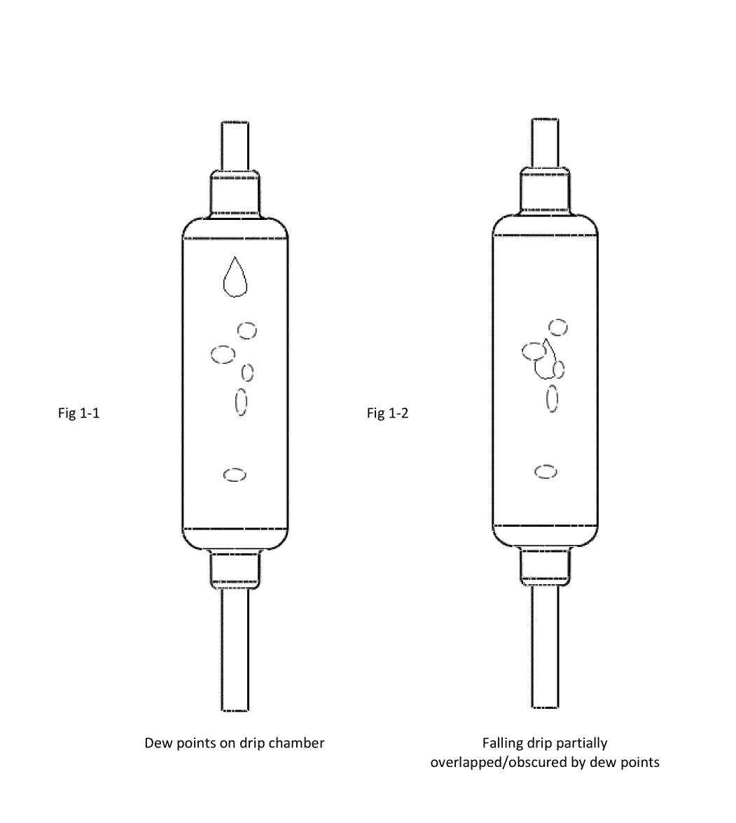

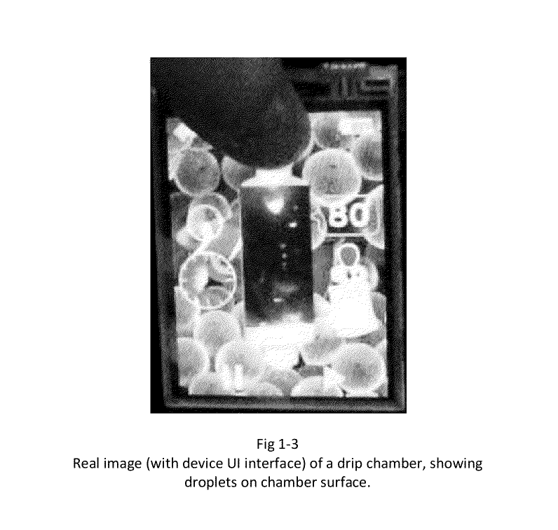

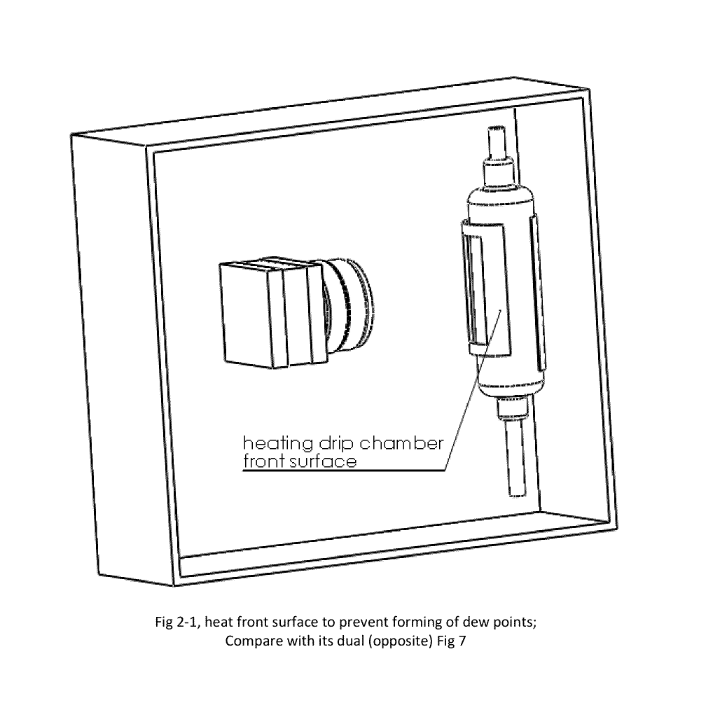

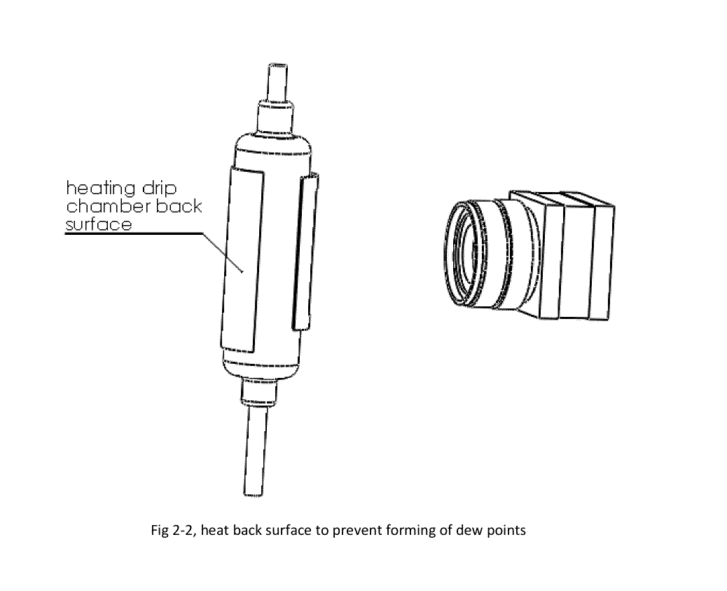

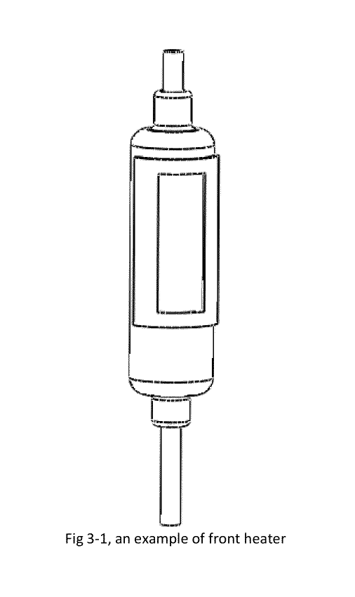

FIG. 1 shows the existence of dew droplets in the captured images.
FIG. 1-1 shows a droplet that has not entered the area of dew droplets.
FIG. 1-2 shows a droplet partially overlapped/obscured by dew droplets.
FIG. 1-3 shows a real image in which both forming drip and dew droplets are can be seen, and the image is surrounded by the GUI of our device.

FIG. 2 shows front and back drip chamber surface heaters.
FIG. 2-1 shows a camera facing the front face of the drip chamber emphasizing the front heater. The front heater has an open window to allow camera to see the drip chamber. Part of the back heater can also be seen. Only five faces of the housing is shown, but it is intended to represent the whole enclosing housing structure.
FIG. 2-2 shows another view of FIG. 2-1, emphasizing the back heater.

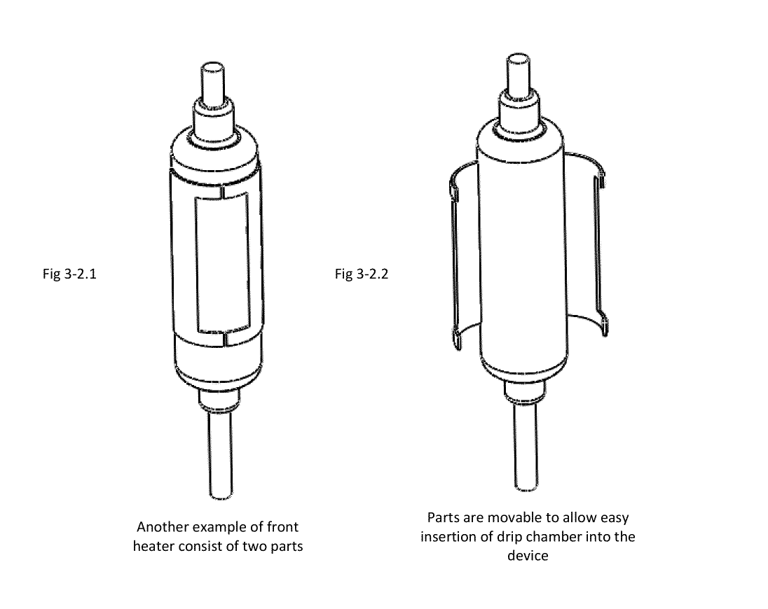

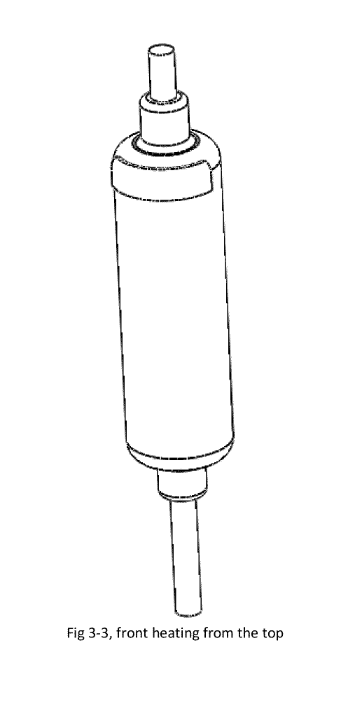

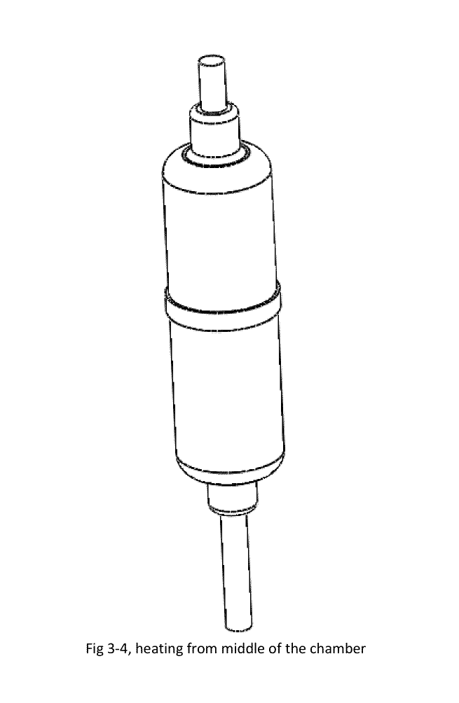

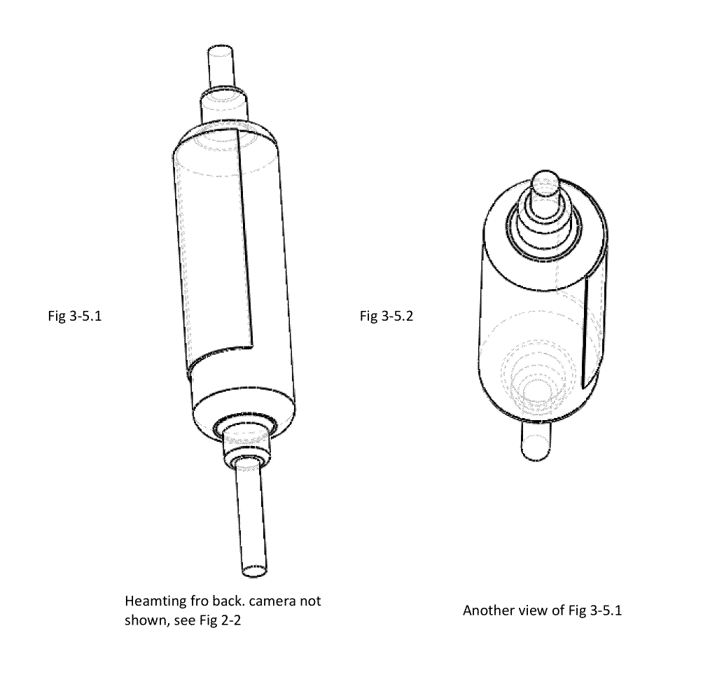

FIG. 3 illustrates different constructions of the heaters (can also be used for cooling after some alterations, please refer to section Cooling and combine Heating/Cooling).
FIG. 3-1 shows an example of the front heater.
FIG. 3-2.1 and FIG. 3-2.2 show an example of the front heater composed of two movable parts
FIG. 3-3 shows a “patch” like front heater at the top.
FIG. 3-4 shows a ring-like heater.
FIG. 3-5.1 and FIG. 3-5.2 show different views of a back heater.

FIG. 4 shows an example of heating by air convection.

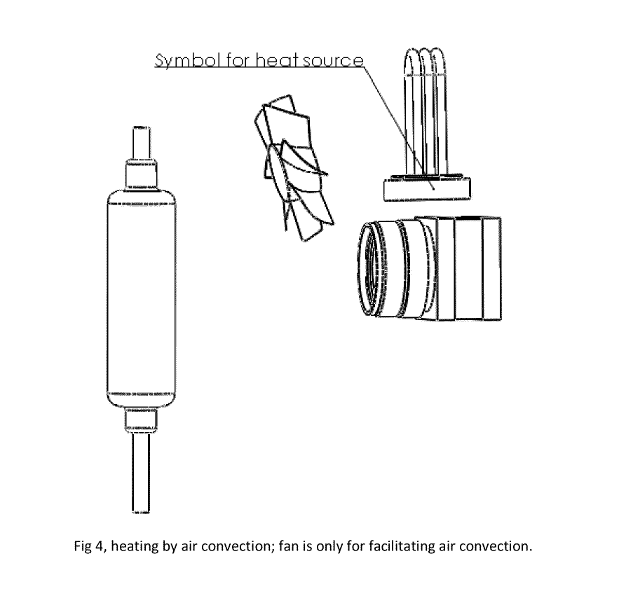

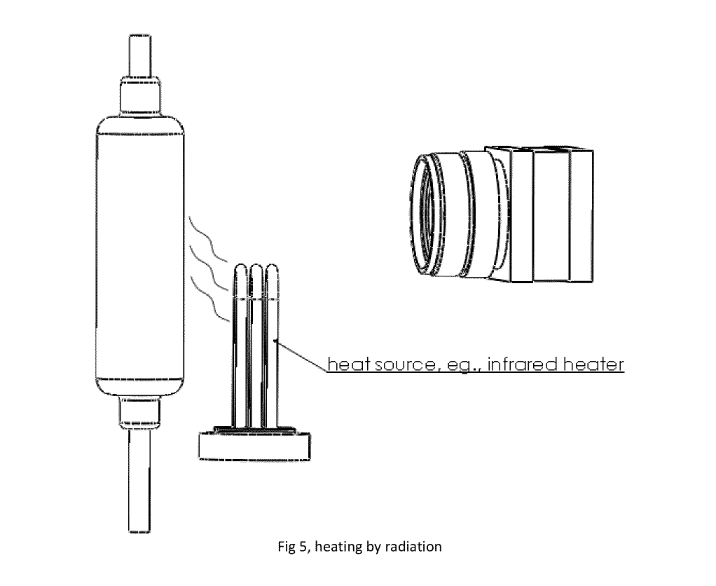

FIG. 5 shows an example of heating by radiation

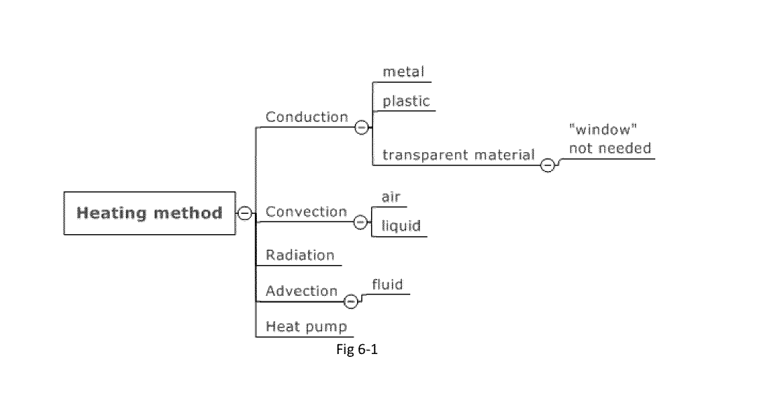

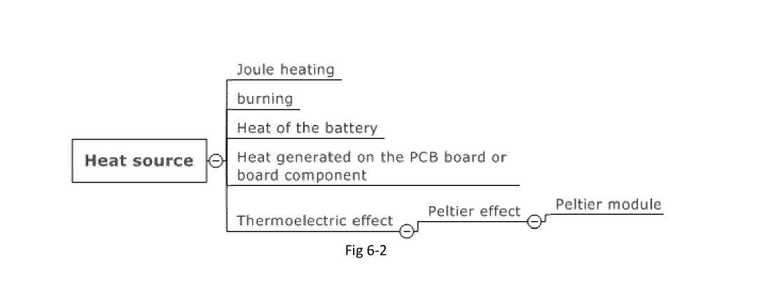

FIG. 6-1 lists some heating methods.

FIG. 6-2 lists some heat sources.

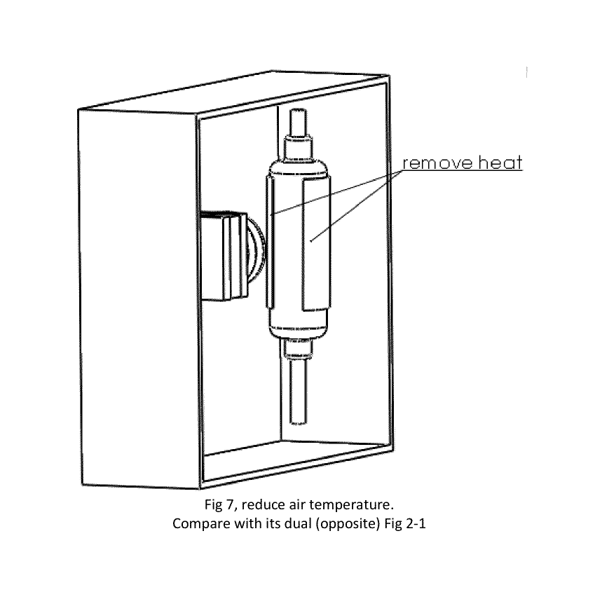

FIG. 7 shows an example of how coolers can be used to lower air temperature inside the drip chamber.

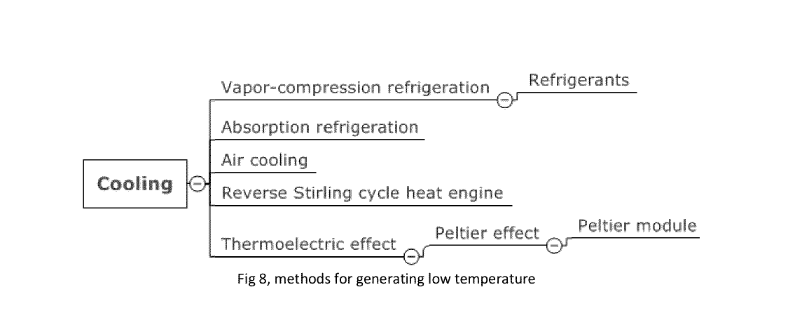

FIG. 8 lists some ways of generating low temperature.

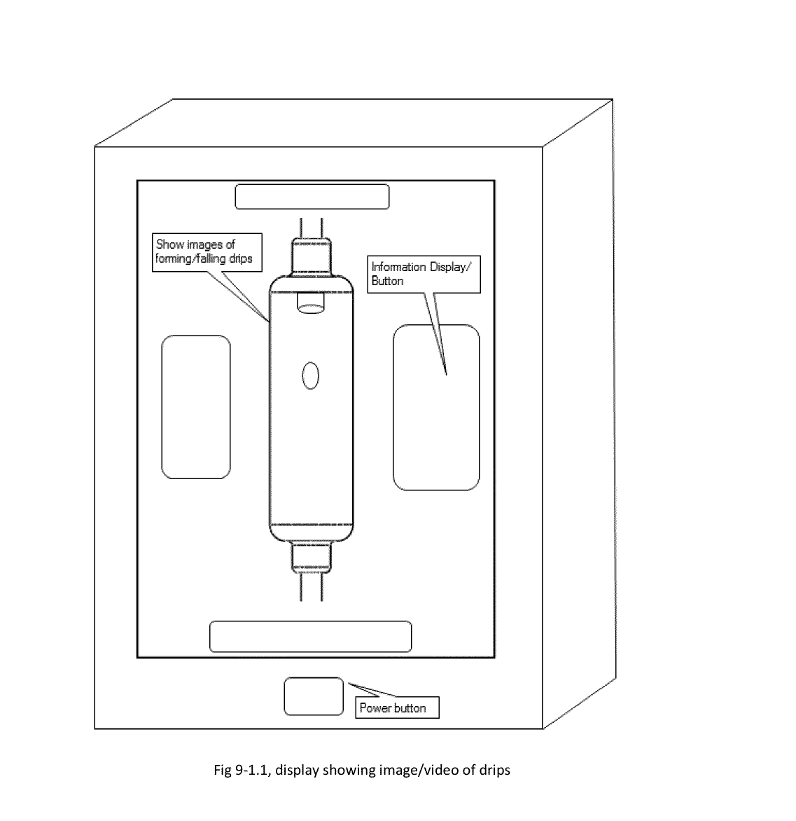

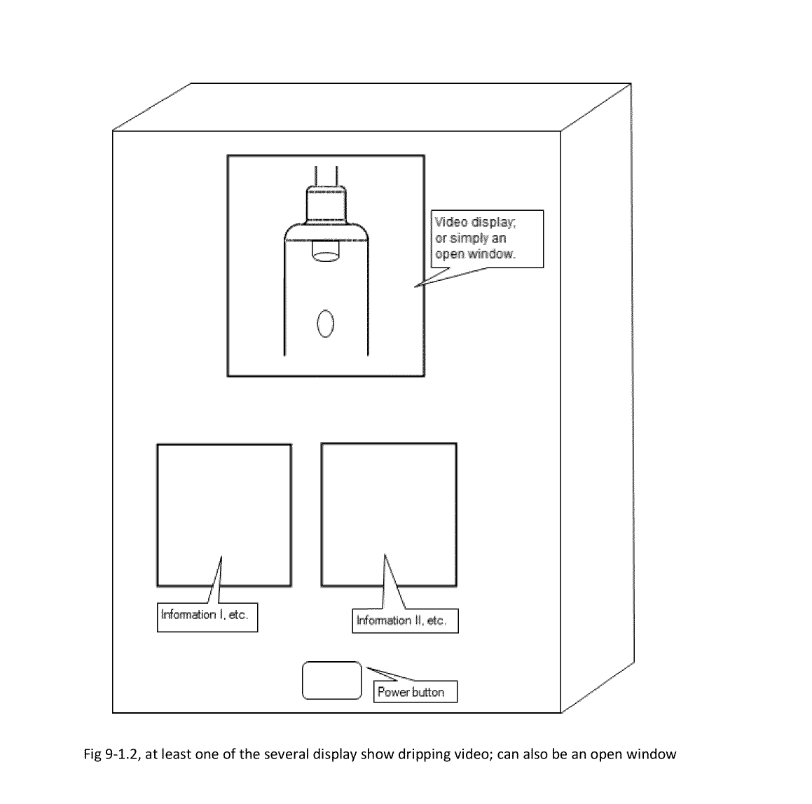

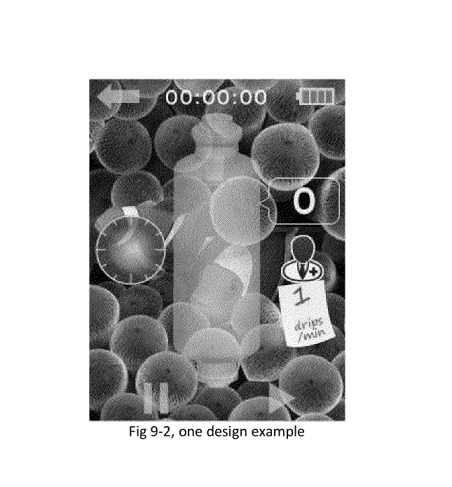

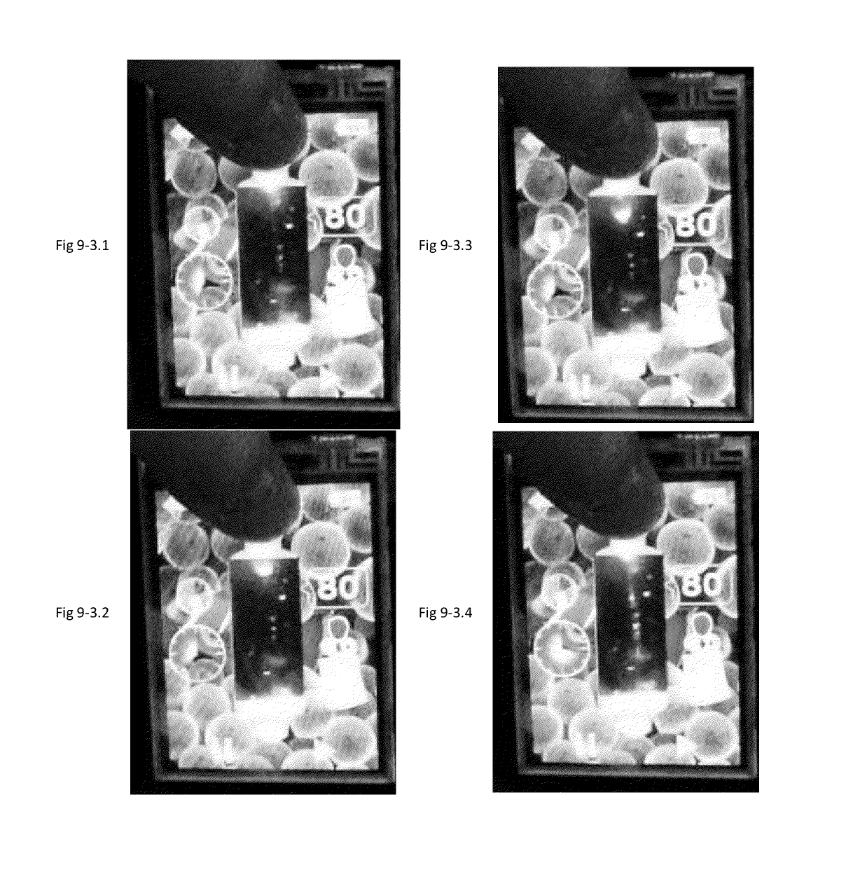

FIG. 9 shows the display of the video of the dripping process on external display.
FIG. 9-1.1 shows an example design of the screen.
FIG. 9-1.2 shows the use of separate displays and one of them if for displaying video of the dripping process.
FIG. 9-2 shows a graphic design example.
FIG. 9-3.1 to FIG. 9-3.4 shows photographs of the running device, and the dripping process can be seen clearly from the monitoring windows.

### DETAILED DESCRIPTION

This application expands on our previous U.S. Ser. No. 12/804,163 and U.S. Ser. No. 13/356,632 applications. Although some parts, such as illumination sources disclosed both in the two previous applications, are not shown explicitly in the present application, it should be understood that they are included by reference, and are implied by necessity. Specification and drawings in this application focus only on the unique and new disclosures.

### Introduction

In this application we describe some unique features of our automatic IV (intravenous therapy) monitoring and controlling device. Please refer to applications U.S. Ser. No. 12/804,163 and U.S. Ser. No. 13/356,632 for previously disclosed details of the invention. This and the two aforementioned applications are all about a new type of IV device we invented. It differs from infusion pump in that it uses computer vision technology to monitor the trajectory (height), size, brightness variation or any periodic signal contained in the video of the IV dripping process and calculate the dripping speed therefrom, then use the speed monitoring information to adjust the thickness of the IV set tube to reach a desired flow rate.

### Handle Dew Droplets by Image Processing Methods

In image processing, just as in any other signal processing application, we always want to have signals of the highest quality and would like to remove noise as much as possible. Images of the IV chamber sometimes contain small dew droplets staying on the surface of the drip chamber, and when trying to identify the actual forming/falling drip we need to distinguish the forming/falling drip from these dew droplets.

This problem is illustrated in FIG. 1-1 and FIG. 1-2. FIG. 1-1 shows dew droplets on the surface of the chamber that is closer to the camera, but the largest drip (can be identified using connected component methods in U.S. Ser. No. 12/804,163) has not yet come to the area containing dew droplets; however in FIG. 1-2, when the falling drip comes into the “dew region”, because the dew droplets are on the chamber closer surface closer to the camera (called “near/front surface” from here, and call the other surface which is farther to the camera the “far/back surface” from here), they could partially block image of the falling drip. As we see in FIG. 1-2, even if we could successfully identify the drip location, we might either calculate a larger drip because the connectivity criteria in U.S. Ser. No. 12/804,163 would merge it with the surrounding droplets, or get a smaller one because parts of the falling drip have been bitten/cut by the dew droplets. It is also possible that the remaining visible area of the falling drips becomes so small because of the blocking or “bitten” effect of the dew droplets so we might mistakenly identify another dew droplet as the largest connected component and hence the drip location.

Although in U.S. Ser. No. 12/804,163 publication [US 2012/0013735 A1] paragraph [0104]-[0108] we have already discussed the essential of the problem: Do a few problematic points invalidate the frequency estimation (Fourier analysis in U.S. Ser. No. 12/804,163, numerous others in U.S. Ser. No. 13/356,632) algorithm? And the answer was that the few noisy points would not change the general periodicity of the signal so that frequency estimation algorithms could always recognize the correct period count. The conclusion has also been experimentally verified by the numerous experiments in U.S. Ser. No. 12/804,163 and U.S. Ser. No. 13/356,632, among which many include the “problematic” signal point of U.S. Ser. No. 12/804,163 FIG. 3D.

We also show a real image of the dew droplets in FIG. 1-3. This drawing was a photograph of our device's user interface (see FIG. 9 and related discussion), and we display what the camera sees on the LCD screen so that patient/nurse could also monitor the dripping speed themselves and compare with the device's result. On the right side of the camera window we see about five dew droplets, but none of them is comparable in size with the forming/falling drip. In fact, in our experiments we have never recorded a case when the dew droplets interfered with the forming/falling drip identification using our connected component algorithm (see U.S. Ser. No. 12/804,163).

In this application we present some additional processing methods that could further improve our result.

Comparing with the forming/falling drips, the dew droplets change their size and location rather slowly. The content in the image sequence (video) due to the forming/falling drips are the fast-changing elements, and the dew droplets are the slow-varying background. A host of techniques can be applied to separate fast-changing information from the slow-varying background. For example we could:
1. Compute only the difference.
   We do this by first capture a frame and use this frame as the “base”. If these images contain dew droplets, subsequent images taken shortly after it will also contain almost the same droplets as in the “base”, and even if there are changes like disappearing or merging of some of the droplets, these changes will not be so significant as long as we keep the time frame (within how long a time frame after the “base” do we take image and compare with the “base”) short. Therefore in general we could assume the background as static, and subtracting from each subsequent image the “base” yields only the difference signal from the “base”, which in general would also be a periodic signal.
2. Use averaging to get the “base”.
   One drawback for randomly taking an image as the “base” is that the “base” might happen to be an image which contains a forming/falling drip, as in the case of FIG. 1-3. There are different ways of computing the “difference”. Because “base” FIG. 1-3 contains a large forming drip, if after taking the signed arithmetic difference between a later I and I_{base}, which is I−I_{base},
   (1) If further take absolute value |I−I_{base}|, we might end up always having the large forming drip area in FIG. 1-3 the largest bright area, which could lead to the wrong identification of a nearly constant drip location as in “base” FIG. 1-3.
   (2) If we truncate the negative part for each pixel pair's difference, then we could still get a periodic signal which is amenable to frequency estimation.
   Nevertheless taking images containing large bright drips as in FIG. 1-3 as the background does not always seem like a logically impeccable method. To ameliorate this, we take a sequence, say 15, of consecutive frames, sum and then average. Since drip change its location across frames, then even for the maximum grayscale value 255, after /15 it becomes 16; and even if during the forming of the drip the position remains almost constant for a number of frames, say 5, dividing by 15 would still bring the area's (near the dripping mouth) grayscale level down to ⅓. In all cases after the averaging we would have the static dew droplet areas remain almost unchanged, but moving contents significantly darkened. By averaging we always get a better background than randomly taking an image.

### Prevent Dew Droplets' Formation or Remove Them

If we want to get perfect signal quality, another approach is to prevent the dew droplet from forming so in image processing stages or remove them so that we get clean images from the beginning. The dew droplets form on drip chamber surface only when the surface temperature is EQUAL or LOWER than the liquid vapor's dew point. Dew point is associated with relative humidity, and as the relative humidity increases, dew point rises and get closer to the current temperature. Therefore if we could keep the temperature of the inner surface of the drip chamber above the dew point, no dew droplets would be able to form on the surface.

FIG. 2-1 illustrates one method to achieve this. We only draw five sides of the housing to reveal the inner arrangement of the camera and the drip chamber, however it should be clearly understood that actual implementation needs to enclose the inner components from all direction to provide an ideal shooting environment for the camera. The camera is drawn on the left, and on the right we see the drip chamber is being wrapped by two bended sheets on the front and back surfaces. Those wrappers are actually heaters providing local, rather than global, heating to the drip chamber. FIG. 2-2 simply gives another view featuring the back heater. The front and back surface heaters can also be seen in FIG. 3-1 and the two subimages of FIG. 3-5.

Let's direct our attention to FIG. 3-1. There is apparently a window intentionally cut in the middle of the wrapper with an obvious purpose of not to block view of the camera. The heat would be applied to the outer side of the drip chamber from the inner (concave) side of the wrapper, reaching the inner side of the drip chamber surface and also by convection (albeit slow on plastic) to the exposed/windowed area. As long as this applied heat keeps the windowed area's temperature above dew point, no dew droplet will be formed and we will always have a clean view.

The specification of U.S. Ser. No. 12/804,163 described in detail why a windowed area would suffice for drip speed measurement. Please refer to that for more information.

Similarly, FIG. 3-5.1 and FIG. 3-5.2 show different views of a back surface heater, corresponding to the annotated part of FIG. 2-2. Using a back surface heater to keep some part of the back side of the inner surface of the drip chamber above dew point could also prevent dew droplets' formation on that area.

Combing the front and back surface heater, we could completely remove the dew droplets shown in FIG. 1-1, FIG. 1-2 and FIG. 1-3. Combined the front and back surface heater with the illumination techniques discussed in U.S. Ser. No. 13/356,632, we would images of almost perfect quality and a guaranteed rock-solid reliability for a medical application.

It should also be noted that we did not specify that both the front and back heather would be simultaneously required. As having been shown by the real image in FIG. 1-3, in many situations (depending on liquid type, drip chamber material, temperature, illumination, camera lens type, etc.) even if dew droplets exist they are still negligible, so the implementation could use just the front or back heater to remove some of the possible dew droplets and leave the remaining few to the treatment of image processing algorithms.

For the front surface heater, it is imperative to leave an open window for camera observation, whereas for the back surface heater this is completely optional. That we are opening a window is based on the presumption that in generally metallic (nontransparent) material will be used for heating due to their good heat conductivity, however if transparent materials can be found which also has acceptable heat conductivity, it can also be used and the window would not be needed.

The shape of the window and the outline of both the front and back heater are also illustrational. Any reasonable shape can be used in real implementation. Please refer to section Experiment and Calculations are important for more information.

Nor is there any requirement that the front and back heater must be separated. We separate them only to make the concepts clearer, n in real implementation one could of course choose whatever combination or make them into an integral whole, as long as the same effect (keep specific area's temperature above dew point) can be achieved.

One might worry whether it would be possible for dew droplets to form on the top inner surface of the drip chamber and flow down to the windowed area (and the corresponding area on the back inner surface). We could add heating directly to the top surface to make it hotter than the dew point, as illustrated by the patch-like heather in FIG. 3-3. The size of the heater in FIG. 3-3 is also purely illustrational and the actual dimension needs to be determined by experiment and calculation. Of course, the top patch in FIG. 1-3 can also be put on the back side.

The necessity of top “patch” like in FIG. 3-3 for preventing dew droplets from forming on the top could only be known after knowing the exact heat/temperature distribution of the drip chamber, please refer to section “Experiment and Calculations are important” for more detail.

FIG. 3-4 shows a “ring” heater surrounding the tube. As long as it can dissipate enough heat to the area of the inner surface to make them hotter than the dew point, it can also be adopted. We include it simply as an example to show the variety of shapes and arrangements the heater could be built like. For the “ring” heather, as long as the camera's actual analysis window (see U.S. Ser. No. 12/804,163) does not stride or overlap the ring area, it would not cause any problem.

In building a real product one has to consider problems like how the drip chamber could be easily inserted/put into the device. A heater like in FIG. 3-1 might need to be moved away first before the drip chamber can be put in, in order to make the use easier we could divide the front heater into two halves, and use simple mechanical structure (for example, hinges driven/rotated by gears) to cause it to open/close before and after putting in the drip chamber, as shown in FIG. 3-2.1 and FIG. 3-2.2.

It is obvious that these mechanical alterations, just as shape of the heaters, are unimportant comparing to their function in keeping temperature of the specific areas of the inner surface above dew point. There are numerous ways to achieve the same effect as in FIG. 3-2.1 or FIG. 3-2.2 but the essentials would be the same.

### Experiment and Calculations are Important

From FIG. 2-1 to FIG. 3.5 we give no specification on the shape, size, multiplicity (how many) or other parameters of the heaters. In real implementation we face some constraints:
1. Excess heat causes humid air temperature to rise and might affect dew point. Although for back heaters like FIG. 3-5.1 it is guaranteed that drip chamber inner surface will be hotter than air because it is in direct contact with the heater, for windowed area like in FIG. 3-1 and FIG. 3-2 the conclusion is less certain because the windowed area is heated by weak conduction of drip chamber's plastic material.
2. From power consumption perspective we should also minimize unnecessary power used on heating. Because we use mechanical systems as disclosed in U.S. Ser. No. 13/019,698 and U.S. Ser. No. 13/356,632 rather than peristaltic pump, the power consumption of the whole IV monitoring and controlling device could be made very low, and in this situation the energy dissipated on heating could be significant when comparing with other parts.

In designing the real product we need to strike a balance between the need of keeping inner surface's specific areas' temperature above dew point, and the considerations above. To reach an optimal design one might need to resort to
(1) Theoretical calculation
(2) Computer simulation
(3) Experiment, such as analyzing heat distribution by thermal imaging

Only after getting quantitative results from the work above could we know the optimal shape, heating temperature, as well as other parameters. Whether we would need the “patch” as in FIG. 3-3 to prevent dew droplets' formation on the top is also a question that can only be answered after knowing the exact heat/temperature distribution.

### Convection, Radiation and Advection

The heaters disclosed above all have direct contact with the drip chamber and therefore heats by conduction. The drip chamber surfaces can also be heated by
1. Convection
   a. Air: as in FIG. 4. The heat source can be of any type and the heat source drawing is only an iconic symbol. The fan is optional and is for facilitating air convection.
   b. Liquid: such as using liquid to carry heat from a source to drip chamber surface.
2. Radiation: as shown in FIG. 5. The heat source drawing is also an iconic symbol and can represent any heat source capable of radiating heat.
3. Advection: It is also possible to implement advection (by air or fluid) to transfer heat to the drip chamber surface with some components.
4. Heat pump: one can also use various types of heat pumps to transfer heat to the specific areas

For these three methods, heating the back surface is not as easy as by direct contact conduction. The calculation/simulation/distribution of heat distribution could also become considerably more difficult than the direct contact conduction heater method, and more effort will be needed in getting the optimal result.

### Monitoring and Controlling Temperature

There are different ways for setting the desired heating temperature. For monitoring temperature of the drip chamber surface, or possibly even the inside, one could use thermocouple (using Seebeck effect, etc.), thermal imaging or else; for controlling temperature one could use a thermostat or else. It should be noted that the choice among these methods, or even future techniques, is unimportant, the important thing is to keep temperatures of specific areas of the drip chamber's inner surface above dew point.

FIG. 6-1 summarized the the heating methods we have discussed so far.

### Heat Source

A vast variety of heat source can be used, the specific choice being unimportant. One should always note that what is important is the purpose of keeping temperatures of specific areas of the drip chamber's inner surface above dew point.

FIG. 6-2 lists some common methods of heating:
1. Ordinarily one can use Joule heating.
2. Oil or other material can also be burned to generate heat
3. The heat of the battery, or heat generated on the PCB board/by components can also be directed the heat the drip chamber.
4. Thermoelectric effect, including using Peltier effect/Peltier module.
5. Other heat sources.

If 3 above is used one has to ensure that the PCB board/components/battery be hot enough and properly preserve the heat when directing it to the heating location. And whatever heat source is used, one must do the calculation/simulation/experiment properly to obtain the optimal parameters.

### Cooling and Combine Heating/Cooling

All the heating methods described so far have cooling as their duals (opposite/complement). This is because our ultimate goal is to keep temperatures of specific areas of the drip chamber's inner surface above dew point, to achieve these we can either increase the temperature of these inner surface areas, or lower the dew point. The dew point can be lowered be lowering the humid air temperature.

Therefore if instead of explicitly transferring heat to those drip chamber surface areas, we may remove heat from the air, or from the liquid which will in turn lead to the lowering of the temperature of the air, and we somehow maintain (or raise, or lower it but keep it still higher than air/liquid) temperature of specific areas of the inner surface of the drip chamber, then they will still be above the dew point.

The wrapper arrangement in FIG. 7 looks exactly like a dual (opposite/complement) of FIG. 2-1. Whereas in FIG. 2-1 heat is explicitly transferred to windowed area and back surface area, in FIG. 7 heat is removed from the two side surfaces which could lower the air temperature inside the chamber. For the drip chamber surface area facing the camera, as well as the it back (far) side, because the heat conduction of the drip chamber's surface is slow, it is possible that the temperature of these areas decrease slower than the air inside the chamber, and in this way we have successfully keep these areas' temperature above the dew point.

If we would like to cool the liquid, we could move the patch-like structure in FIG. 3-3 to the bottom and it would effectively lower the liquid temperature down, and consequently temperature of the air above the liquid.

Because the dual (opposite/complementary) relationship between cooling and heating, all the heating methods listed in FIG. 6-1 and/or discussed above, as well as all arrangements from FIG. 2 to FIG. 5, can be used on cooling after straightforward modifications.

It is also obvious that cooling/heating can be applied simultaneously to create the relative difference so that the temperature of specific areas of the inner surface of the drip chamber is above the dew point.

### Generating Cold Temperature

FIG. 8 lists common methods for generating low temperature. These method include
1. Vapor-compression refrigeration (Refrigerants)
2. Absorption refrigeration
3. Air cooling
4. Reverse Stirling cycle heat engine
5. Thermoelectric effect (Peltier effect, Peltier module)

All methods can be used, however method 5's implementation relatively is the easiest among the listed method above. No matter whatever cooling method is used, one must do the calculation/simulation/experiment properly to obtain the optimal parameters.

### Applicability

Our fundamental method of keeping the temperature of specific areas of the inner surface of the drip chamber above dew point could improve image quality for all types of periodic measurements including those disclosed in U.S. Ser. No. 12/804,163 and U.S. Ser. No. 13/356,632, no matter it is trajectory (height) based, drip size based, brightness variation based or others. For the methods disclosed by Cheng U.S. Ser. No. 12/791,885 Intravenous Drip Monitoring Method And Related Intravenous Drip Monitoring System, whose basic idea is equivalent to our average gray level measurements in U.S. Ser. No. 12/804,163 (FIG. 4I and FIG. 4J and the corresponding specification text), the removal of dew droplets is actually more important because for this class of brightness variation methods we usually do not get the information as rich as in the trajectory (drip height) or size measurement, and these brightness variation measurements are more susceptible to interferences from the dew droplets. Using the dew droplet removal/prevention methods in our present invention would significantly improve the signal quality particularly for the brightness variation class methods, as well as for other more sophisticated methods.

### Display Dripping Video on the Display

In our series of applications (U.S. Ser. No. 12/825,368, U.S. Ser. No. 12/804,163, U.S. Ser. No. 13/019,698, U.S. Ser. No. 13/356,632) we enclose the drip chamber inside the device and blocks external lights to create an ideal shooting environment for the computer vision system. However from the patient/nurse's standpoint, without being able to see the actual droplets coming down from the dripping mouth they tend to be skeptical on the calculated speed as being displayed on the external display (see FIG. 1 in U.S. Ser. No. 12/804,163). Other applications such as Cheng Ser. No. 12/791,885 did not mention the use of an external display. CN201110955Y of Enmin Song displays only numeric data, but not the video of the dripping process as in our present disclosure.

We believe a visual display of the dripping process inside the device is essential to our user's experience and to their confidence with this device. Therefore as in the design drawing of FIG. 9-1.1, we specifically dedicate an area on the display for showing the video of the dripping process. Although preferably the display is a touchscreen LCD through which all input/output can be exchanged graphically and interactively, there is no inherent requirement that LCD be the only choice (CRT display may also work; new display technologies might soon emerge, etc.). What is important is that we display at least an area of the camera's view on the display which contains at least some part of the drip's forming/falling process, allowing the user to see the actual dripping process and count the periods.

For aesthetic purposes we might also represent some part of the dripping chamber by UI graphic just as shown in FIG. 9-2, and display only a small window of the camera's video which contains the enough information to the human observer (user).

FIG. 9-3.1 to FIG. 9-3.4 show four frames of video being displayed on the LCD screen of our real device. The formation of the drip in FIG. 9-3.1 to FIG. 9-3.3 as well as the eventual fall in FIG. 9-3.4, are very clear to the user and they could easily count the speed themselves and compare with the algorithm result (shown as 80 drips/mean in the callout window on the right).

Because inside the microprocessor the display and video input module typically use different buffer and memory space, when implementing this one needs to properly copy the input video frames to the display output buffer, and the details would depend on the specific choice of the processor and peripheral ICs.

It is also possible to use separate display devices, for example one LCD (CRT, etc.) module to show only the video of the dripping process, and other modules to show numeric monitoring information (possibly by simpler and cheaper display technology). This type of arrangement is shown in FIG. 9-1.2.

What is important here is provide a means to the user to monitor the actual dripping process of the drip chamber enclosed inside the device.

Another implementation is to open a window on the devices housing, which can either be a permanent opening or a window that can be covered by a lid/cover, and allow the user to monitor the inside by seeing into that window area (possibly after moving the lid/cover). This arrangement is also shown in FIG. 9-1.2.

We claim:

1. The use of image processing methods to remove the dew droplets from the backgrounds for images captured on an IV dripping chamber.

2. Apparatus to keep the temperature of some areas of the inner surface of the drip chamber to be higher than the dew points of the humid air inside the drip chamber.

3. A means to display video of the dripping process inside the device to external display(s).
